(19) United States
(12) Patent Application Publication
(10) Pub. No.: US 2007/0112286 A1
(43) Pub. Date: May 17, 2007

(54) Device to measure acceleration and deceleration of hip rotation

(76) Inventor: Robert Prichard, Tiburon, CA (US)

Correspondence Address: Robert Prichard, 4 Tara Hill Road, Tiburon, CA, 94920, US

(21) Appl. No.: 11/256,517

(22) Filed: Nov. 17, 2005

(51) Int. Cl. A61B 5/103

(52) U.S. Cl. 600/595

(57) ABSTRACT

Hip rotation measuring device with an accelerometer, a micro-controller, a printed circuit board, a memory chip, a power supply such as batteries, an on-off switch, a start-stop recording switch, an acceleration direction switch an audible sound generator, a readout display such as an LCD display, standard support electronics such as resistors capacitors and regulators for operating the electronic components, a case to house the components, and a belt clip or other standard attachment device fixed to the outside of the case so that when the components are combined within the case and when the case is attached by the clip to an article of clothing that is in close proximity to a moving body part such as a hip, the readout display can show magnitude of acceleration or deceleration of hip movement of the user. The device can measure other body part acceleration and deceleration such as hand, foot, shoulders.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of electronic measuring devices and more specifically to a hip rotation measuring device.

Research has shown that increasing hip rotation acceleration dramatically improves racquet head speed in tennis, bat speed in baseball, club speed in golf, hand speed in boxing, throwing speed in baseball and football, and even peak hand force in swimming. Better athletes are known to have higher rates of hip acceleration and deceleration during their sport than poorer performing athletes. Until now, there has not been an easy, economical way for an athlete to measure his or her hip rotation acceleration or deceleration. Prior methods have required expensive and time consuming set-ups with body markers, computers and video cameras.

The prior art includes numerous methods of using electronic measuring means to evaluate range of motion of various parts of the human body, such as Crandall et al in his U.S. Pat. No. 4,922,925 that evaluates range of motion in the hand and wrist. Or Misevich et al U.S. Pat. No. 5,216,817 that measures the geometries and axes of rotation of parts of the human body. However, none of the prior art specifically teaches a portable device that measures acceleration or deceleration of a body part.

Since hip rotation is so important to any sport involving hip rotation, it would be very helpful to an athlete seeking to improve his or her performance to be able to easily, quickly and inexpensively measure his or her hip rotation acceleration and deceleration. It would also be helpful to an athlete to have immediate feedback about the magnitude of the acceleration and deceleration of other body parts such as the hands, feet, knees, arms or shoulders.

### BRIEF SUMMARY OF THE INVENTION

The primary object of the invention is to provide a device that measures and records the magnitude of acceleration and deceleration of hip rotation in a human being during a sport activity.

Another object of the invention is to provide a hip rotation measuring device that is worn by the user and gives immediate feedback regarding hip rotation acceleration and deceleration.

Another object of the invention is to provide a hip rotation measuring device that give immediate feedback to the wearer or instructor about the magnitude of the acceleration and deceleration of other body parts such as the hand, foot, shoulders and knee during a sport activity.

A further object of the invention is to provide a hip rotation measuring device that gives immediate feedback, via a transmitter and receiver, to a broadcaster during a sporting event about the magnitude of acceleration and deceleration of hip rotation or other body parts.

Yet another object of the invention is to provide a hip rotation measuring device that is inexpensive to manufacture.

Still yet another object of the invention is to provide a hip rotation measuring device that is light weight, portable and easily attached to the user so that it does not adversely affect the performance of the wearer during a sport activity.

Another object of the invention is to provide a hip rotation measuring device that is small enough and enclosed in a waterproof casing so that it can be attached to a person's swim suit so that it can be used while swimming.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a hip rotation measuring device comprising: an accelerometer, a micro-controller, a printed circuit board, a memory chip, a power supply such as batteries, an on-off switch, a start-stop recording switch, an acceleration direction switch an audible sound generator, a readout display such as an LCD display, standard support electronics such as resistors capacitors and regulators for operating said electronic components, a case to house said components, and a belt clip or other standard attachment device fixed to the outside of said case so that when said components are combined within said case and when said case is attached by said clip to an article of clothing that is in close proximity to a moving body part such as a hip, said readout display can show magnitude of acceleration or deceleration of hip movement of the user.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the invention.

FIG. 2 is a schematic view of the electronic portion of the invention.

FIG. 3 is a rear perspective view of the invention.

FIG. 4 is a front view of the invention.

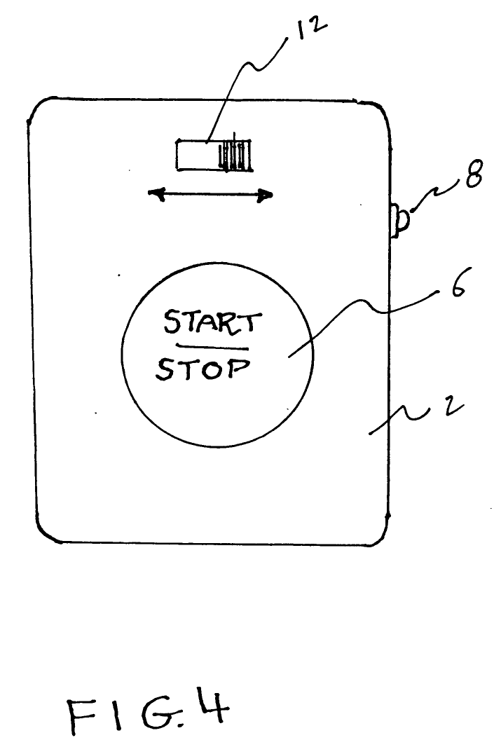

FIG. 5 is a top view of the invention.

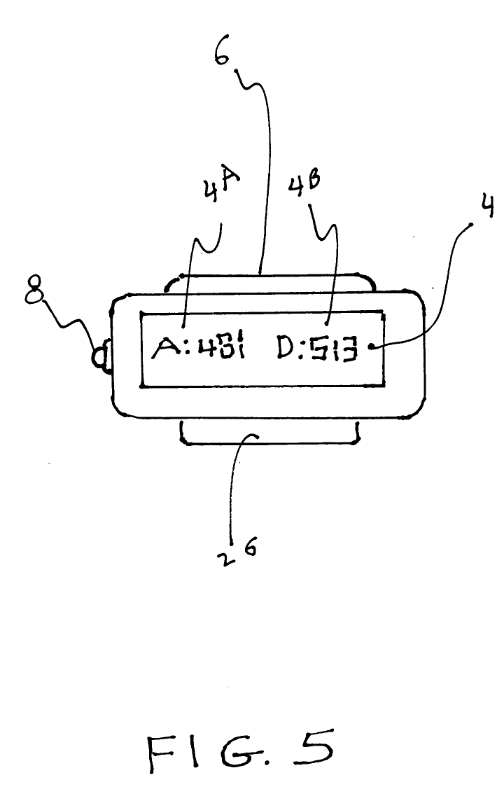

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

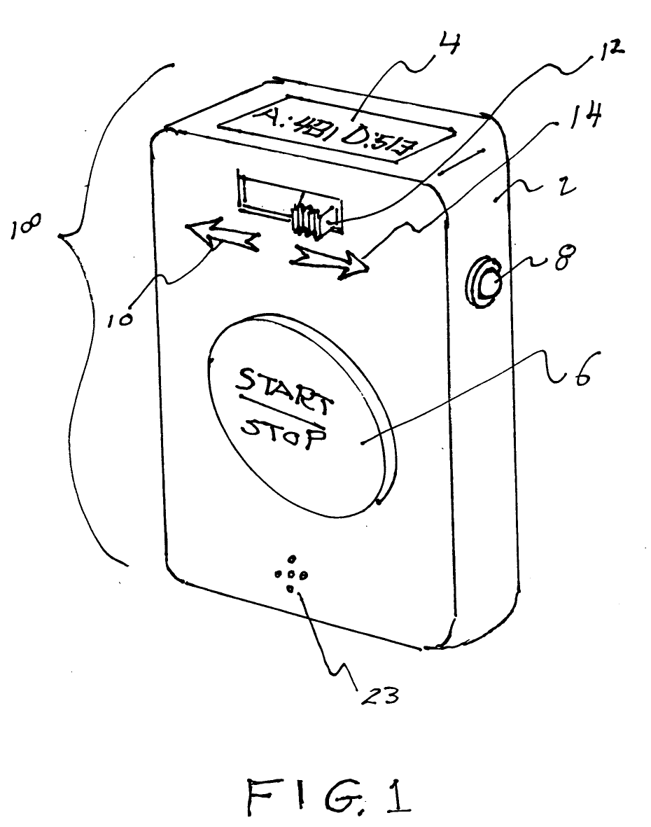

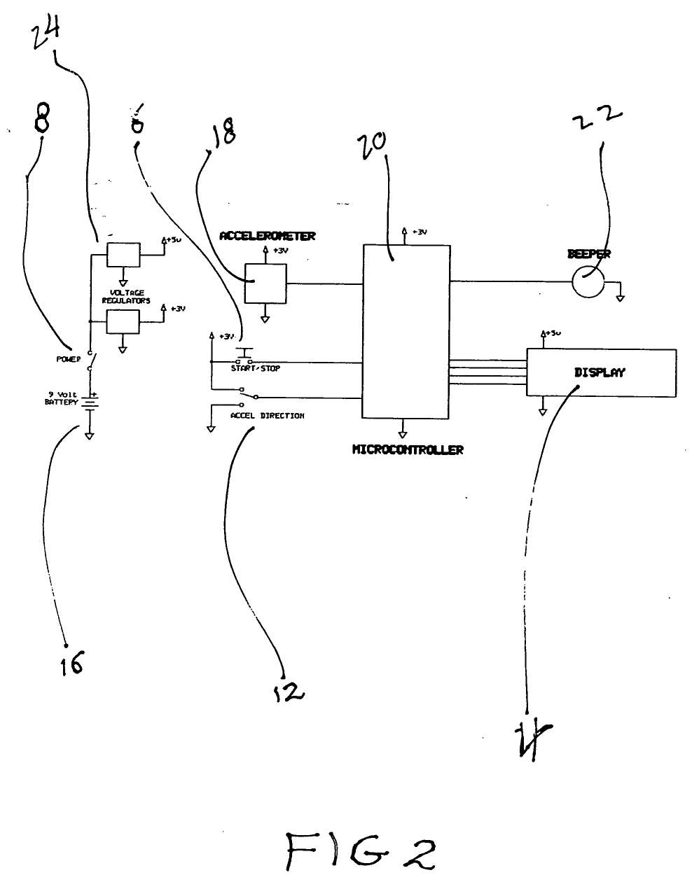

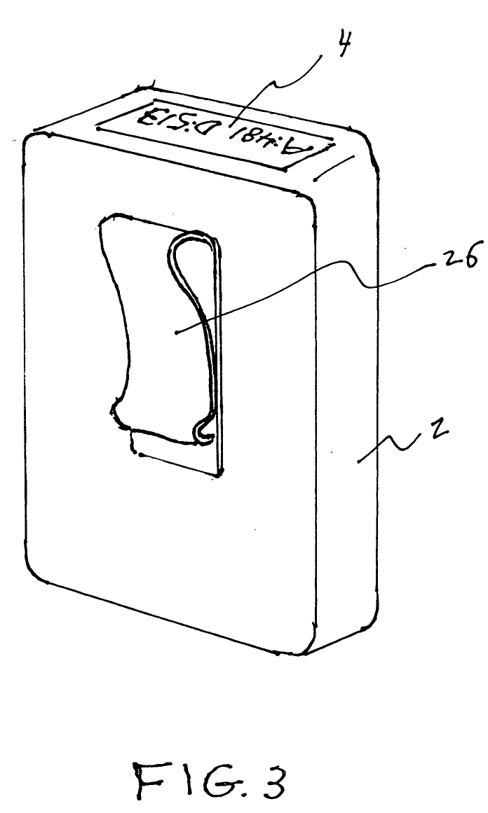

referring now to FIG. 1 we see a perspective view of the invention 100. The outer case 2 is smaller than a pack of cigarettes and therefore portable and capable of attaching to a person's clothing by a clip 26 or other standard fastening device as shown in FIG. 3. Push button 6 starts and stops the process of measuring acceleration or deceleration. Slide switch 12 lets the user select direction of acceleration measurement as shown by arrow 10 or by arrow 14. Push button 8 turns the entire unit on or off. Apertures 23 allow sound from audio tone generator 22. Case 2 houses components that are shown in FIG. 2. Accelerometer 18 is attached to signal detecting and amplifying micro-controller 20 which includes a memory chip and powered by a battery power source 16. The circuit is further controlled by direction switch 12, on-off switch 8 and start-stop recording switch 6. The entire circuit is mounted on a standard printed circuit board, not shown. Additional support electronics 24 are standard components such as resistors, capacitors and voltage regulators. The micro-controller 20 sends information to display portion 4, preferably an LCD display, thereby showing the user the magnitude of acceleration and deceleration during a time period as defined by the time elapsed between pressing button 6 to start the recording of measurements and pressing button 6 again to stop the recording of measurements. An audio tone generated by standard tone generating device 22 sounds loudly at the start of recording, beeps softly during the duration of the recording period and then sounds loudly again and the end of the recording period.

During use, the user attaches the device 100 to his or her belt or pants top by clip 26 and moves the direction switch 12 in the direction he or she will be rotating his or her hips during a sport activity such as swinging a bat, tennis racquet, golf club, racquetball racquet, table tennis racquet, cricket bat, or throwing a baseball, football, javelin, discus or throwing a punch or kicking a soccer ball, football, or martial arts kick. The device 100 is switched on by pressing the on-off switch 8 to the “on” position. Then the start button 6, which also can be called the recording button, is pressed, at which time the device starts to record acceleration and deceleration in the direction of the direction switch 12. The user then presses button 6 again thereby stopping the recording process and initiating the display of maximum values during the elapsed time period for acceleration and deceleration. The display is cleared and the device starts to record again when the recording button 6 is pressed again. Pressing the on-off switch to the “off” position turns off the device 100.

The device can be easily modified with the addition of an audio speaker or earphone to provide an auditory message regarding magnitude of acceleration or deceleration instead of, or as well as, the visual readout 4 already described. Additionally, the information could be transmitted by standard transmitter and receiver means to a remote location such as a TV or radio announcer's booth or external display.

Additionally, the device 100 can be modified in size and shape to be worn on the wrist like a wristwatch to display readings for the hand during a punch or throw or it can be strapped to the ankle or foot to display reading during a kick, or inserted into a special pocket or attached to the back of a shirt, vest or coat to record the rotation of the shoulders during a swing, throw or punch. Finally, the entire unit 100 can be enclosed in a waterproof housing for use during swimming activity.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. Hip Rotation Measuring Device comprising:
an accelerometer;
a micro-controller;
a printed circuit board;
a memory chip;
a power supply such as batteries;
an on-off switch;
a start-stop recording switch;
an acceleration/deceleration direction switch
an audible sound generator;
a readout display such as an LCD display;
standard support electronics such as resistors capacitors and regulators for operating said electronic components;
a case to house said components; and
a belt clip or other standard attachment device fixed to the outside of said case
so that when said components are combined within said case and when said case is attached by said clip to an article of clothing that is in close proximity to a moving body part such as a hip, said readout display can show magnitude of acceleration or deceleration of hip movement of the user.

2. Hip Rotation Measuring Device as claimed in claim 1 wherein said measuring device can measure other body part acceleration and deceleration such as hand, foot, shoulders or knee.

3. Hip Rotation Measuring Device as claimed in claim 1 wherein said measuring device can be housed inside a waterproof enclosure for use during swimming activity.

4. Hip Rotation Measuring Device as claimed in claim 1 wherein said device is inexpensive to manufacture.

5. Hip Rotation Measuring Device as claimed in claim 1 wherein said measuring device is light weight and portable.

6. Hip Rotation Measuring Device as claimed in claim 1 wherein said measuring device is easy to operate.

7. Hip Rotation Measuring Device as claimed in claim 1 wherein said measuring device gives immediate feedback to the wearer or instructor about the magnitude of acceleration and deceleration of hip rotation during a sport activity.

8. Hip Rotation Measuring Device as claimed in claim 1 wherein said measuring device can include a standard transmitting portion that can give immediate feedback to a TV or radio broadcaster or an external display during a sporting event about the magnitude of acceleration and deceleration of hip rotation or other body parts such as the hand, foot, shoulders or knee.

9. Hip Rotation Measuring Device as claimed in claim 1 wherein said measuring device can provide immediate feedback to the wearer or care giver about the magnitude of acceleration and deceleration of body parts during the course of rehabilitation.
